(19) United States
(12) Patent Application Publication
(10) Pub. No.: US 2023/0158323 A1
(43) Pub. Date: May 25, 2023

(54) WEARABLE PULSED ELECTROMAGNETIC FIELD SENSING DEVICE

(71) Applicant: Regenesis Biomedical, Inc., Scottsdale, AZ (US)

(72) Inventors: Manuel J. FERRER HERRERA, Mesa, AZ (US); Erica TASSONE, Gilbert, AZ (US); Frank E. CONTRERAS, Phoenix, AZ (US); Meredith SMITH, Mesa, AZ (US); Ryan BINETTE, Scottsdale, AZ (US)

(21) Appl. No.: 18/051,834

(22) Filed: Nov. 1, 2022

Related U.S. Application Data
(60) Provisional application No. 63/274,473, filed on Nov. 1, 2021.

(51) Int. Cl. A61N 2/02; A61N 2/00

(52) U.S. Cl. CPC A61N 2/02; A61N 2/004

(57) ABSTRACT

A method and apparatus for detecting a pulsed electromagnetic field (PEMF) therapy is disclosed. In some embodiments, the apparatus may include a PEMF sensor to detect electrical signals from a site on the patient’s body that are remote from an applied PEMF therapy; the electrical signal may be detected as therapeutic PEMF waveforms are delivered to a patient, and may be proportional to the applied PEMF treatment. A clinician may monitor the PEMF therapy that is delivered to the patient through the separate device. The apparatus may be wearable and may include a bracelet, ring, belt, band, or strap that enables the patient to comfortably wear the apparatus when receiving PEMF therapy.

### CLAIM OF PRIORITY

This patent application claims priority to U.S. Provisional Pat. Application No. 63/274,473, filed on Nov. 1, 2021, titled “WEARABLE PULSED ELECTROMAGNETIC FIELD SENSING DEVICE” and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Pulsed electromagnetic fields (PEMF) have been described for treating therapeutically resistant problems of both the musculoskeletal system as well as soft tissues. PEMF typically includes the use of low-energy, time-varying magnetic fields. For example, PEMF therapy has been used to treat non-union bone fractures and delayed union bone fractures. PEMF therapy has also been used for treatment of corresponding types of body soft tissue injuries including chronic refractory tendinitis, decubitus ulcers and ligament, tendon injuries, osteoporosis, and Charcot foot. During PEMF therapy, an electromagnetic transducer coil is generally placed in the vicinity of the injury (sometimes referred to as the “target area”) such that pulsing the transducer coil will produce an applied or driving field that penetrates to the underlying tissue.

Treatment devices emitting magnetic and/or electromagnetic energy offer significant advantages over other types of electrical stimulators because magnetic and electromagnetic energy can be applied externally through clothing and wound dressings, thereby rendering such treatments completely non-invasive. Moreover, published reports of double-blind placebo-controlled clinical trials utilizing a RF transmission device (Diapulse) suggest that this ancillary treatment device significantly reduces wound healing time for open, chronic pressure ulcers as well as for surgical wounds. Studies using Dermagen, a magnetic device manufactured in Europe which produces a low frequency magnetic field, have demonstrated significant augmentation of healing of venous stasis ulcers.

Although PEMF therapies have shown promise, accurately monitoring delivered PEMF treatment may be difficult. Relying on indirect information, for example provided by the patient, is prone to inaccuracies regarding duration, time of day, number of treatments, and so forth. Accurate reporting of delivered therapies may allow a clinician to adjust several treatment parameters. It is also difficult for patients themselves to know when energy has been applied in an appropriate level, because PEMF typically cannot be sensed. Thus, it would be very beneficial to more accurately track PEMF treatment delivered to patients.

### SUMMARY OF THE DISCLOSURE

In general, described herein are pulsed electromagnetic field (PEMF) detection apparatuses (e.g., devices and systems, including PEMF therapy systems) and methods for detecting an applied PEMF therapy. In some examples these apparatuses and methods may be part of the applied PEMF applicator system and/or method of applying the PEMF therapy. The PEMF detection apparatuses described herein may include a PEMF sensor that can detect PEMF therapy delivered to a patient. The detection of delivered PEMF therapy may advantageously be used by a clinician to verify that the patient has received a PEMF treatment, help track clinical trials, and enable the clinician to tailor PEMF therapies. These apparatuses (devices, systems, etc.) and methods may also help track patient dosing and treatments, and my therefore help with compliance and reimbursement issues. Other uses are possible.

Described herein are methods for detecting a PEMF treatment provided to a patient. The method may include attaching a first device (e.g., a PEMF detector) to the patient and/or having the patient hold the first device, wherein the PEMF detector that includes at least one PEMF sensor (e.g., including an antenna), detecting, with the PEMF sensor, an electrical signal due to an applied PEMF treatment delivered to a patient, and determining that the electrical signal received is due to a PEMF treatment (e.g., by comparing the amplitude and/or frequency to an applied PEMF signal), and transmitting, to a second device, the PEMF treatment level in response to determining that the PEMF treatment level is greater than the threshold.

As described herein, the PEMF treatment may be provided by a second device (PEMF applicator). A PEMF applicator may communicate with the PEMF detector directly or indirectly. In some examples the method and apparatus may include analyzing the detected signal by comparing with a threshold; the threshold may be associated with a PEMF detection threshold.

The method may further include transmitting, to the second device, patient biometric data. The patient biometric data may include at least one of body temperature, heart rate, pulse rate, skin capacitance, blood oxygen levels, or a combination thereof. In some cases, PEMF treatment may be verified via the detected electrical signal level and/or frequency and/or the patient biometric data.

Also described herein are systems and apparatuses including a PEMF therapy device coupled to a PEMF applicator. The second device may be at least one of a smart phone, a tablet computer, a laptop computer, a smart watch, or a server computer.

The first device disclosed in the method may be is a wearable device worn by the patient. The first device may include an armband, a ring, a belt, a wrist band, a necklace, a strap, a band, an anklet, a disposable patch, a smart watch adaptor, a watch adaptor, or a combination thereof. In some cases, the first device may include a wireless transmitter. The wireless transmitter may transfer wireless data in accordance with a Bluetooth, Wi-Fi, or cellular protocol. In some variations, the first device may be powered, at least in part, by harvested radio frequency (RF) energy. The RF energy may be provided, at least in part, by pulsed electromagnetic fields provided by a PEMF applicator.

The PEMF treatment level of the methods disclosed herein may be proportional to pulsed electromagnetic fields provided by a PEMF applicator.

Also described herein are pulsed electromagnetic field (PEMF) systems. The PEMF system may include a first device configured to be worn by a patient; that includes at least one PEMF sensor. The PEMF sensor may be configured to detect a PEMF treatment level delivered to the patient, determine that the PEMF treatment level is greater than a threshold, and transmit to a second device the PEMF treatment level in response a determination that the PEMF treatment level is greater than the threshold.

As described herein, the detected PEMF treatment level of the PEMF system may be provided by the second device. In some cases, the detected PEMF treatment level may be provided by a PEMF applicator. The threshold of may be associated with a PEMF detection threshold.

As described herein, the first device of the PEMF system may be further configured to transmit patient biometric data to the second device. In some cases, the patient biometric data includes at least one of body temperature, heart rate, pulse rate, skin capacitance, blood oxygen levels, or a combination thereof. In some other cases, a PEMF treatment may be verified via the detected PEMF treatment level and the patient biometric data. The first device may be a wearable device worn by the patient. In some variations, the first device may include an armband, a ring, a belt, a wrist band, a necklace, a strap, a band, an anklet, a disposable patch, a smart watch adaptor, a watch adaptor, or a combination thereof. In some other variations, the first device may include a wireless transmitter. The wireless transmitter may transfer wireless data in accordance with a Bluetooth, Wi-Fi, or cellular protocol. The first device may be powered, at least in part, by harvested radio frequency (RF) energy. The RF energy may be provided, at least in part, by pulsed electromagnetic fields provided by a PEMF applicator.

As described herein the second device of the PEMF system may be a PEMF therapy device coupled to a PEMF applicator. The second device may be at least one of a smart phone, a tablet computer, a laptop computer, a smart watch, or a server computer.

As further described herein, the PEMF treatment level may be proportional to pulsed electromagnetic fields provided by a PEMF applicator.

Also described herein are pulsed electromagnetic field (PEMF) detection apparatuses that may include a PEMF sensor configured to detect PEMF treatment levels received by a patient, a comparison unit configured to determine that a detected PEMF treatment level to is greater than a threshold, and a wireless transmitter configured to transmit the detected PEMF treatment level to a second device in response to a determination that the detected PEMF level is greater than the threshold.

As described herein the wireless transmitter of the PEMF detection apparatus may be configured to transfer wireless data in accordance with a Bluetooth, Wi-Fi, or cellular protocol. Furthermore, the PEMF detection apparatus may include a sensor to detect patient biometric data. The patient biometric data may include at least one of body temperature, heart rate, pulse rate, skin capacitance, blood oxygen levels, or a combination thereof. As further described herein the PEMF detection apparatus may verify a PEMF treatment via the detected PEMF treatment level and the patient biometric data.

As described herein, the second device may be a PEMF therapy device coupled to a PEMF applicator. In some cases, the second device may be at least one of a smart phone, a tablet computer, a laptop computer, a smart watch, or a server computer.

As further described herein, the detected PEMF treatment level may be proportional to pulsed electromagnetic fields provided by a PEMF applicator. The PEMF detection apparatus may be configured to be to be powered, at least in part, by harvested radio frequency (RF) energy. The RF energy may be provided, at least in part, by pulsed electromagnetic fields provided by a PEMF therapy device.

Also described herein are non-transitory computer-readable storage medium storing instructions that, when executed by one or more processors of a pulsed electromagnetic field (PEMF) system. Execution of the non-transitory computer-readable storage medium may cause the PEMF system to detect, via a first device, a PEMF treatment level delivered to a patient, determine that the PEMF treatment level is greater than a threshold, and transmit, to a second device, the PEMF treatment level in response a determination that the PEMF treatment level is greater than the threshold.

As described herein, the detected PEMF treatment level may be provided by the second device. In some cases, the PEMF treatment may be provided by a PEMF applicator. As also described herein, the threshold may be associated with a PEMF detection threshold.

In some variations, the first device may be further configured to transmit patient biometric data to the second device. Furthermore, the patient biometric data may include at least one of body temperature, heart rate, pulse rate, skin capacitance, blood oxygen levels, or a combination thereof. A PEMF treatment may be verified via the detected PEMF treatment level and the patient biometric data.

In some cases, the second device may be at least one of a smart phone, a tablet computer, a laptop computer, a smart watch, or a server computer.

For example, described herein are methods for detecting a pulsed electromagnetic field (PEMF) treatment provided to a treatment site on a patient, the method comprising: detecting, with a PEMF sensor that is held or worn at a detection site on the patient that is distal from the treatment site, an electrical signal; determining that the electrical signal correspond to a PEMF treatment applied to the treatment site; and transmitting, to a second device, data characterizing the PEMF treatment in response to determining that the electrical signal corresponds to a PEMF treatment level.

Any of these methods may include determining that the electrical signal corresponds to the PEMF treatment applied to the treatment site by determining that the electrical signal is greater than a threshold. These methods may also include determining that the electrical signal corresponds to the PEMF treatment applied to the treatment site comprises determining that the electrical signal has a frequency within a predefined range.

In any of these apparatuses, the method may include transmitting data characterizing the PEMF treatment by transmitting one or both of: a PEMF treatment level and a PEMF treatment duration. The PEMF treatment level is proportional to pulsed electromagnetic fields provided by a PEMF applicator.

Any of these methods may include attaching the PEMF sensor to the patient. In some examples the PEMF sensor may be held or worn at the detection site on the patient, and may include detecting, with the PEMF sensor that is worn on the patient’s arm, hand or wrist.

The PEMF sensor that may be held or worn at the detection site on the patient, and may detect, with a PEMF sensor that is worn on a garment worn by the subject.

The PEMF sensor may detect an electrical signal corresponding to a PEMF signal with the PEMF sensor that is an armband, a ring, a belt, a wrist band, a necklace, a strap, a band, an anklet, disposable patch, a smart watch adaptor, a watch adaptor, or a combination thereof.

In any of these methods PEMF treatment may be concurrently applied at a treatment site while the applied PEMF may be detected or confirmed by a PEMF detector that is held (or worn) at a site that is distal from the PEMF application site.

Any of these methods may include detecting and/or transmitting patient biometric data. The patient biometric data may include at least one of body temperature, heart rate, pulse rate, skin capacitance, blood oxygen levels, or a combination thereof.

In some examples the second device may be a PEMF therapy device coupled to a PEMF applicator. The second device may be at least one of a smart phone, a tablet computer, a laptop computer, a smart watch, or a server computer. The PEMF sensor may be powered, at least in part, by harvested radio frequency (RF) energy provided by a PEMF applicator.

Also described herein are pulsed electromagnetic field (PEMF) detection apparatuses, that may include: a PEMF sensor comprising one or more coils; a housing configured to enclose the PEMF sensor, further configured to be held or worn by the patient; and a processor configured to determine that a patient is receiving a PEMF treatment, the processor, a memory coupled to the processor, the memory configured to store computer-program instructions, that, when executed by the processor, perform a computer-implemented method comprising: detecting an electrical signal with the PEMF sensor; determining that the electrical signal correspond to a PEMF treatment applied to a treatment site on the patient’s body; and transmitting, to a second device, data characterizing the PEMF treatment in response to determining that the electrical signal corresponds to the PEMF treatment.

The apparatus may include one or more sensor configured to detect biometric data when the apparatus is held or worn by the patient. The patient biometric data may include at least one of body temperature, heart rate, pulse rate, skin capacitance, blood oxygen levels, or a combination thereof.

Any of these apparatuses may include a processor configured to determine that the electrical signal correspond to the PEMF treatment applied to the treatment site on the patient’s body by determining that the electrical signal is greater than a threshold. The processor may be configured to determine that the electrical signal correspond to the PEMF treatment applied to the treatment site on the patient’s body by determining that the electrical signal has a frequency within a predefined range.

The transmitting data characterizing the PEMF treatment may include one or both of: a PEMF treatment level and a PEMF treatment duration. Transmitting data characterizing the PEMF treatment may comprise transmitting a magnitude and/or a frequency of the detected electrical signal.

The housing may be configured to be held or worn by the patient’s arm, hand or wrist. For example, the housing may be configured to be worn on a garment worn by the subject. In some examples the housing is configured to be a bracelet, an armband, a ring, a belt, a wrist band, a necklace, a strap, a band, an anklet, a disposable patch, a smart watch adaptor, a watch adaptor, or a combination thereof.

Also described herein are pulsed electromagnetic field (PEMF) detection apparatus comprising: a PEMF sensor configured to be worn or held at a detection site to detect an electrical signal indicative of a PEMF treatment received by a patient at a site remote from the detection site; a comparison unit configured to determine that a detected electrical signal is greater than a PEMF threshold and to determine data characteristic of the PEMF treatment; and a wireless transmitter configured to transmit the data characteristic of the PEMF treatment to a second device in response to a determination that the detected PEMF level is greater than the threshold. The data characteristic may indicate that the PEMF treatment is detected (yes/no), e.g., may include an indication that PEMF therapy has been or is being applied. The data characteristic of the PEMF treatment may include an indication of the level and/or frequency PEMF therapy has been or is being applied.

In general, the PEMF detection apparatus may be configured to transfer wireless data in accordance with a Bluetooth, Wi-Fi, or cellular protocol.

The PEMF detection apparatus may include one or more sensors to detect patient biometric data, such as (but not limited to) at least one of: body temperature, heart rate, pulse rate, skin capacitance, blood oxygen levels, or a combination thereof.

The detected electrical signal may be proportional to pulsed electromagnetic fields provided by a PEMF applicator.

Any of the PEMF detection apparatuses descried herein may be configured to be powered, at least in part, by harvested radio frequency (RF) energy.

Also described herein are non-transitory computer-readable storage medium storing instructions that, when executed by one or more processors of a pulsed electromagnetic field (PEMF) system, cause the system to: detect, with a PEMF sensor that is held or worn at a detection site on the patient that is distal from the treatment site, an electrical signal; determine that the electrical signal correspond to a PEMF treatment applied to the treatment site; and transmit, to a second device, data characterizing the PEMF treatment in response to determining that the electrical signal corresponds to a PEMF treatment level.

The PEMF treatment level may be provided by the second device. For example, the PEMF treatment level may be provided by a PEMF applicator.

The non-transitory computer-readable storage media may be configured to transmit patient biometric data to the second device, includes at least one of body temperature, heart rate, pulse rate, skin capacitance, blood oxygen levels, or a combination thereof. For example, the non-transitory computer-readable storage medium may verify PEMF treatment via the detected electrical signal, including in some examples by using the patient biometric data.

All of the methods and apparatuses described herein, in any combination, are herein contemplated and can be used to achieve the benefits as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the methods and apparatuses described herein will be obtained by reference to the following detailed description that sets forth illustrative embodiments, and the accompanying drawings of which:

FIG. 1 is a diagram of an example of a PEMF treatment system.

FIG. 2 is a block diagram of a PEMF sensing device.

FIG. 3 is a flowchart depicting an example of one method for detecting PEMF therapy delivered to a patient.

FIG. 4 shows a block diagram of a PEMF therapy device.

FIG. 5 schematically illustrates an example of a PEMF sensor subsystem apparatus as described herein.

FIG. 6 schematically illustrates various possible sub-systems of a PEMF sensor subsystems apparatus.

FIG. 7 schematically illustrates an example of a PEMF sensor subsystem as described herein.

### DETAILED DESCRIPTION

A pulsed electromagnetic field (PEMF) sensing device may include a sensor configured to detect PEMF treatment provided to a patient from a PEMF applicator. The PEMF sensing device may be wearable. For example, the PEMF sensing device may include a wearable band or the like that may allow the patient to comfortably wear the device remote from a PEMF treatment area and still detect that a PEMF therapy is being applied. The PEMF sensing device may detect one or more PEMF treatment aspects including PEMF strength and duration.

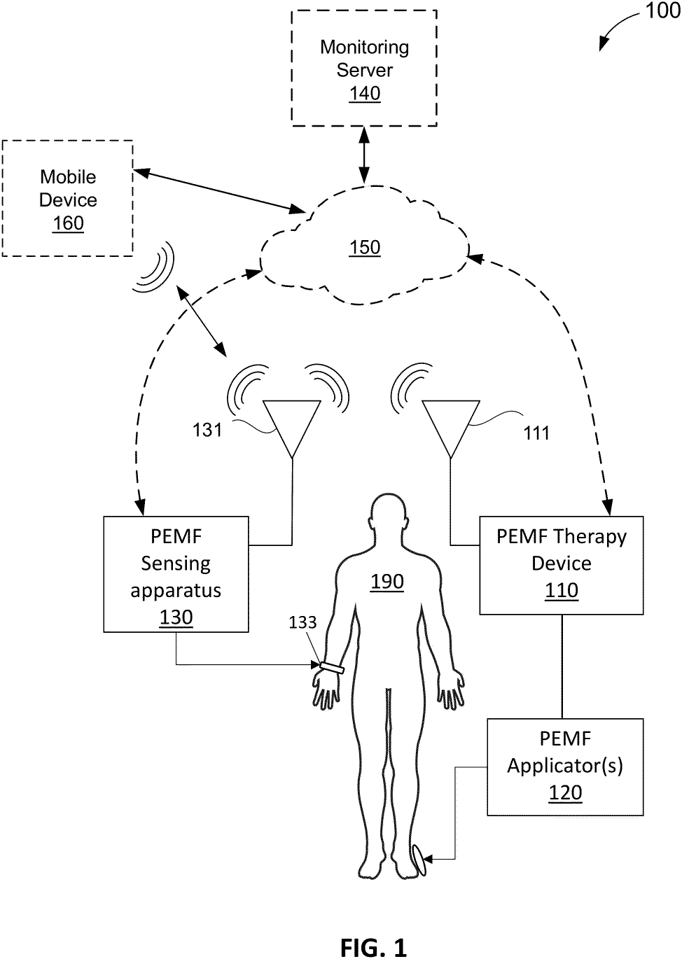

The PEMF sensing or detection apparatus (e.g., system, device, etc.) may detect an electrical signal to determine if the detected electrical signal that is detected is characteristic of a PEMF signal applied to a remotely located treatment site. For example, a patient may wear a bracelet or wrist PEMF sensing device (or a ring, armband, belt, etc.) while a PEMF therapy apparatus applied (via an applicator) PEMF therapy to the patient’s foot or leg, as shown in FIG. 1 discussed in greater detail below. The sensed electrical signal may arise directly or indirectly from the applied PEMF therapy; surprisingly this electrical signal, which may be proportional to the applied PEMF signal, may be detected when the sensor is worn or held by a part of the body that is remote from the treatment site.

In practice, the PEMF detecting apparatuses described herein may record, transmit and/or analyze a detected signal. For example, the apparats may detect an electrical signal and analyze it to determine if the magnitude and/or frequency information is consistent with a therapeutic PEMF therapy. The apparatus may determine the duration of an applied PEMF signal.

In some examples, the PEMF sensing device may be wirelessly coupled to a second device. The second device may record (e.g., log) PEMF information provided by the PEMF sensing device that, in turn, may be reviewed by a clinician. In some examples, the second device may log detected PEMF treatment times, duration and detected PEMF strength. The PEMF sensing device may use Bluetooth, Wi-Fi, internet of things (IoT), or any other feasible wireless technology to communicate with the second device.

The second device may be a PEMF therapy device, a remote PEMF analysis unit, a cell phone, a tablet computer, a laptop, or any other feasible device. Since the PEMF sensing device directly monitors and detects PEMF treatment, inaccuracies based on patient self-reporting are avoided.

Particular implementations of the subject matter described in this disclosure can be implemented to realize one or more of the following potential advantages. For example, the described techniques may enable a clinician to verify or validate clinical trials concerning the application of PEMF therapy. In addition, or alternatively, PEMF sensing data and patient biometric data (collected with the PEMF sensing data) may be used to monitor and track a patient’s response to PEMF therapy. In some cases, a clinician may review the patient’s response and modify the PEMF therapy accordingly.

FIG. 1 is a diagram of an example of a PEMF treatment system 100, according to some examples. A PEMF treatment system 100 may include a PEMF therapy device 110, a PEMF applicator 120, and a PEMF sensing device 130. The PEMF therapy device 110 may be used to deliver pulsed electromagnetic fields to a patient through one or more PEMF applicators, such as the PEMF applicator 120. Although only one PEMF applicator 120 is shown (coupled to the patient 190 on the patient’s foot), in other examples, the PEMF treatment system 100 may include any feasible number of PEMF applicators 120. The pulsed electromagnetic fields may provide a therapeutic effect to the patient in a non-invasive manner. In some examples, the pulsed electromagnetic fields may upregulate cytokines, collagen, alpha SMA, FGF and other markers associated with wound healing. In still other examples, the pulsed electromagnetic fields may treat inflammation and tissue remodeling associated with a predicted or pending diabetic foot ulcers and/or pressure ulcers.

Separately or (optionally) as a part of a PEMF system, a PEMF sensing device 130 may detect PEMF treatment (e.g., detect PEMF therapy as detected PEMF levels) administered to the patient through, for example, the PEMF applicator 120. In some embodiments, the PEMF sensing device 130 may include one or more PEMF sensors within a held or worn device 133 adapted to receive an electrical signal from the patient that may be related to the electromagnetic fields delivered to the patient by the PEMF applicator 120. One example of a sensor may include one or more loops of a conductor (e.g., antenna) that may have a voltage induced through the loop. Surprisingly the sensors described herein may detect the PEMF signal when worn or held by the body, even when positioned on the body at a location that is distant from the PEMF applicator. The sensor may detect an electrical signal that is passed through the body. If the PEMF sensor is not held or worn by the patient receiving the treatment it may not detect the applied PEMF energy.

Thus, a PEMF sensor may include an antenna. In some cases, the PEMF sensing device 130 may also include an analog-to-digital converter (ADC) (not shown) to measure and quantify the induced voltage. Additionally, the PEMF sensing device 130 may include any number of amplifiers and/or filters. The PEMF sensing device 130 may optionally include a comparison device (not shown). The comparison device may compare detected (sensed) PEMF therapy levels to a predetermined threshold to determine whether PEMF therapy is actively being received. In this manner, the PEMF sensing device 130 may be able to discriminate between PEMF therapy levels and possible background levels. The detected (sensed) PEMF levels may be proportional to pulsed electromagnetic fields provided by the PEMF applicator 120. In some cases, the predetermined threshold may be a PEMF detection threshold related to a minimum detection level associated with actively receiving PEMF therapy.

The PEMF sensing device 130 and the PEMF therapy device 110 may both include wireless communication units (not shown). For example, a wireless transceiver of the PEMF therapy device 110 may be coupled to antenna 111 and the wireless transmitter of the PEMF sensing device 130 may be coupled to antenna 131. Together, the wireless communication units of the PEMF therapy device 110 and the PEMF sensing device 130 and the antennas 111 and 131 may enable data to be exchanged. For example, PEMF levels detected by the PEMF sensing device 130 and/or PEMF levels greater than a predetermined threshold detected by the PEMF sensing device 130 may be transmitted to the PEMF therapy device 110. In some cases, the PEMF therapy device 110 may log the detected PEMF levels (detected electromagnetic fields greater than a threshold). The therapy logs may include time and duration associated with the detected therapy.

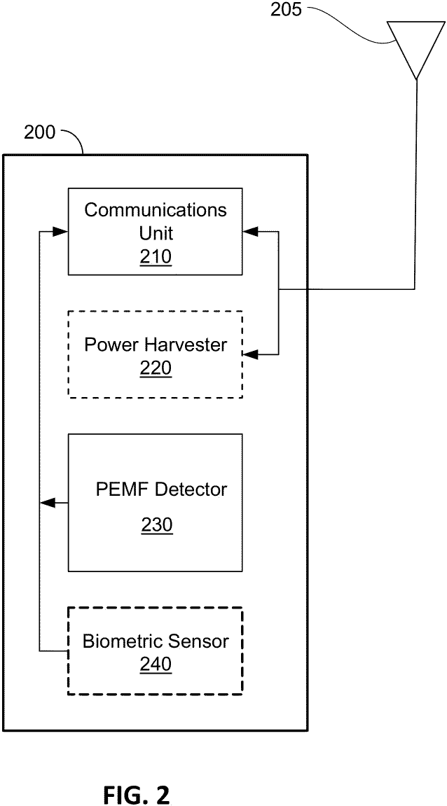

Some PEMF sensing devices 130 may include biometric sensors (not shown) that can monitor heart rate (pulse rate), body temperature, skin capacitance, blood oxygen levels, and the like. In this manner patient biometric data may also be transmitted to the PEMF therapy device 110. The patient biometric data may be logged in the PEMF therapy device 110 similar to the PEMF level information. In some variations, the patient biometric data and/or the PEMF level information may be used to verify a patient’s identity and/or confirm that a particular patient is receiving PEMF treatment. For example, therapy logs and biometric data logs may be used to confirm that PEMF therapy of particular time and/or duration has been delivered to a patient. Thus, therapy logs and biometric data logs may be used verify various therapy trials. Biometric sensors are described in more detail in conjunction with FIG. 2. Patient biometric data may also be transmitted to the PEMF therapy device 110.

The PEMF sensing device 130 may be worn by the patient. For example, the PEMF sensing device may include a band, elastic band, or similar device to, at least temporarily, affix the PEMF sensing device 130 to the patient. In some other variations, the PEMF sensing device 130 may be a patch that may include an adhesive to attach the PEMF sensing device 130 to the patient. The PEMF sensing device 130 may be reusable or disposable based at least in part on choice of materials, construction, or cost/benefit targets.

In some examples, the PEMF detection apparatus is held by the patient. for example the PEMF apparatus may be configured to be coupled to a smartphone, as, e.g., a phone case or the like. The phone case may include a wireless communication with the phone and may use the phone processor to analyze, store and/or transmit the received signal. Alternatively the PEMF detection apparatus includes its own processor for analyzing, storing and/or transmitting data, including data characterizing a detected PEMF signal.

The PEMF therapy device 110 may optionally be coupled directly or indirectly to a monitoring server 140. For example, the monitoring server 140 may be coupled to the PEMF therapy device 110 through a network 150. The network 150 can be any feasible network including a generic communication network such as the Internet. In this manner, data from the PEMF sensing device 130 can be transmitted to the monitoring server 140 (e.g., through the PEMF therapy device 110). The monitoring server 140 may be optional as illustrated by the dashed lines in FIG. 1.

A clinician may access the monitoring server 140, the PEMF therapy device 110, and/or the PEMF sensing device 130 (also referred to herein as a PEMF detecting device, a PEMF detector, or the like) to access PEMF sensing data and/or patient biometric data. As described above, PEMF sensing data (e.g., detected PEMF levels) and the patient biometric data may be used in a variety of ways. For example, PEMF sensing data and/or patient biometric data may be used as data to verify or validate clinical trials. In another example, PEMF sensing data and/or patient biometric data may be used to monitor and track a patient’s response to PEMF therapy. In still another example, PEMF sensing data and patient biometric data may be used to verify that a particular patient has received PEMF treatment. After reviewing this data, the clinician may adjust a frequency (number of times per day, for example) or PEMF strength to tailor the delivered PEMF therapy to the patient. In some instances, the clinician may review (monitor) PEMF treatment information and/or biometric information in “real time” as the PEMF treatment is being provided to the patient.

In another variation, a mobile device 160 may wirelessly communicate with the PEMF sensing device 130. For example, the mobile device 160 and the PEMF sensing device 130 may communicate via Bluetooth, Wi-Fi, or any other feasible communication protocol. A mobile device 160 may be a smart phone, a tablet computer, a laptop computer, a smart watch, a fitness tracker, or any other feasible mobile device. In some cases, the mobile device 160 may receive the PEMF sensing data and/or the patient biometric data and may transmit this data to the monitoring server 140. In some other variations, the mobile device 160 analyze PEMF sensing data and/or patient biometric data and/or allow a clinician access to the data to perform their own analysis.

In one example, the PEMF sensing device 130 may function as a smart watch or a smart watch adaptor. That is, the detected PEMF data and the detected patient biometric data may be transmitted to from the PEMF sensing device 130 to a smart watch (e.g., the mobile device 160). In turn, the smart watch may transmit PEMF data and patient biometric data to the PEMF therapy device 110, the monitoring server 140, or any other feasible device.

The PEMF sensing device 130 may include a power harvesting unit (not shown). The power harvesting unit may harvest sufficient energy from radio-frequency (RF) energy to provide power to the PEMF sensing device 130. RF energy may be provided by PEMF treatment, nearby Wi-Fi, Bluetooth or any other feasible RF sources. In some variations, the PEMF sensing device 130 may include a battery, a rechargeable battery or super capacitor. The power harvesting unit may provide power (e.g., charge) the rechargeable battery or super capacitor.

In general, any of the apparatuses described herein may include a controller and/or one or more processors which may be configured to compare detected PEMF treatment levels to a threshold and/or record PEMF treatment information including the detected PEMF treatment levels, time of day associated with the PEMF treatment and in some cases biometric patient data. In addition, the controller and/or processors may be configured to transmit and/or receive wireless data.

FIG. 2 is a block diagram of an example of a PEMF sensing device 200, according to some examples. The PEMF sensing device 200 may include a communications unit 210, an optional power harvester 220, a PEMF detector 230, and biometric sensors 240. The PEMF sensing device 200 may be one example of an implementation of the PEMF sensing device 130 of FIG. 1.

The communications unit 210 may be coupled to an antenna 205. The communications unit may provide wireless communications functionality. For example, the communications unit 210 may wirelessly transmit (and in some cases receive) data between other devices. The communications unit 210 may include devices, circuits, components, and the like to transmit and/or receive data in accordance with Bluetooth, Wi-Fi, cellular, or any other feasible communication protocol.

A power harvester 220, which may also be coupled to the antenna 205, may receive RF energy and convert the RF energy into power, such as direct current power. The power from the power harvester 220 may provide power for the PEMF sensing device 200. In some cases, the power from the power harvester 220 may provide charge for a battery, super capacitor or the like for the PEMF sensing device 200.

The PEMF detector 230 (which may include a comparator) may detect the presence of PEMF therapies provided or directed to a patient. For example, the PEMF detector/comparator 230 may include a PEMF sensor that may detect and/or sense PEMF associated waveforms received by the patient by detecting an electrical signal. Thus, the PEMF detector/comparator 230 may generate PEMF sensing data. For example, sensed electrical signal may be proportional to pulsed electromagnetic fields provided by a PEMF applicator. In some cases, the PEMF detector 230 may compare the level of any received electrical signals to a predetermined threshold. Alternatively or additionally the frequency information on the detected electrical signal may be compared to determine if it falls within the range of frequencies associated with an applied PEMF signal (e.g., having a carrier frequency of about 27.12 MHz or signals of an about 42 µsec pulses with a period of 1 KHz). If the level and/or frequency of the received signal is within a target range (e.g., a threshold range or above a threshold value), then the PEMF detector 230 determines that the patient is receiving a PEMF therapy and may monitor the application for duration, energy applied, etc. Some or all of this information may be referred to herein as data characterizing the PEMF treatment.

In some variations, the PEMF detector/comparator 230 may include an output such as a light emitting device (e.g., lamp or light emitting diode) and/or a vibration device (not shown). When the level of the PEMF signal detected, then the light may be turned on and/or a vibration produced to indicate to the patient that a PEMF therapy is being received.

The biometric sensor 240 may be an optional component of the PEMF sensing device 200 (as depicted by the dashed lines). The biometric sensor 240 may generate patient biometric data that includes one or more of heart rate (pulse rate), body temperature, skin capacitance, and blood oxygen levels of the patient.

The PEMF detector/comparator 230 and the biometric sensor 240 may be coupled to the communications unit 210. In this manner, PEMF information and biometric data may be transmitted from the PEMF sensing device 200 to any other suitable device. Suitable devices may include PEMF therapy devices (such as the PEMF therapy device 110 of FIG. 1), mobile devices including smart phones, smart watches, laptop computers, tablet computers, fitness trackers and the like, or remote computing devices (such as the monitoring server 140).

In some examples, the PEMF sensing data and/or patient biometric data may be used as data to verify or validate clinical trials. In another example, PEMF sensing data and/or patient biometric data may be used to monitor and track a patient’s response to PEMF therapy. In still another example, PEMF sensing data and patient biometric data may be used to verify that a particular patient has received PEMF treatment. After reviewing this data, the clinician may adjust a frequency (number of times per day, for example) or PEMF strength to tailor the delivered PEMF therapy to the patient. In some instances, the clinician may review PEMF treatment information and/or biometric information in “real time” as the PEMF treatment is being provided to the patient.

In some variations, the PEMF sensing device 200 may be worn by a patient. Thus, the PEMF sensing device 200 may include an armband, a ring, a belt, a wrist band, a necklace, a strap, a band, an anklet, or the like to enable the patient to comfortably wear the PEMF sensing device 200. The PEMF sensing device 200 may be disposable or reusable. The PEMF sensing device 200 may include an adhesive that enables the PEMF sensing device 200 to be temporarily coupled (affixed) to the patient.

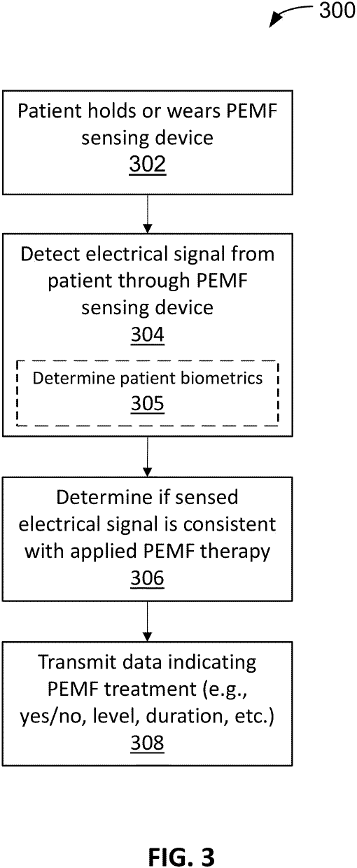

FIG. 3 is a flowchart depicting an example of one method 300 for detecting PEMF therapy delivered to a patient. Some examples may perform the operations described herein with additional operations, fewer operations, operations in a different order, operations in parallel, and some operations differently. The operations herein are described as being performed by the PEMF treatment system 100 of FIG. 1 ease of explanation. Persons having skill in the art will recognize that the operations described herein can be performed by any feasible device or processor that may be configured to receive and/or detect the conditions described herein and perform and/or deliver the therapies described herein.

In FIG. 3, the method 300 may begin as the PEMF sensing device 130 is held and/or attached to a patient 302. For example, the PEMF sensing device may include a strap, wristband or the like and may be worn by the patient. In some cases, the PEMF sensing device 130 may take the form of a patch that may be attached through adhesive to the patient or the patient’s clothing.

Next, 304 the PEMF sensing device 130 may detect PEMF treatment. For example, a sensor within the PEMF sensing device 130 may sense or detect an electrical signal that correlates with applied PEMF waveforms received and conducted by the patient. In some variations, the PEMF sensing device 130 may optionally determine the patient’s biometric data 305. For example, the PEMF sensing device 130 may include one or more biometric sensors that can determine a patient’s heart rate (pulse rate), body temperature, skin capacitance, blood oxygen levels, or any other feasible patient biometric information. A patient’s biometric data may be used to confirm a patient’s identity and/or confirm that a particular patient is receiving a PEMF treatment.

Next, 306, the PEMF treatment system 100 determines whether the detected electrical signal is consistent with an expected PEMF treatment. For example, the electrical signal received may be compared to a frequency range and/or threshold and/or an amplitude range and/or threshold (e.g., greater than a predetermined threshold). For example, the PEMF sensing device 130 may determine a detected level of PEMF treatment. The detected level may be compared to a predetermined threshold. In some cases, a detected PEMF level greater than a threshold indicates that PEMF therapy is being detected. If the detected PEMF level is greater than the threshold, a light (LED) or haptic (vibration) device of the PEMF sensing device 130 may be activated. Thus, a light or vibration may be provided to the patient to indicate that a PEMF therapy is being received.

Next, 308, the detected data characterizing the PEMF treatment (e.g., detected, PEMF level, duration of treatment, etc.) may be determine, stored, and/or transmitted. In some cases, the detected data characterizing the PEMF treatment is transmitted to any feasible device to enable access to the PEMF data by a clinician. Patient biometric information gathered 305 may be transmitted along with PEMF level information. In some instances, data characterizing the PEMF treatment and patient biometric information may be transmitted to a separate device such as the PEMF therapy device 110, the mobile device 160 or the monitoring server 140. Thus, comparison of detected PEMF levels to a predetermined threshold may be performed at the receiving device.

In some variations, PEMF level information and patient biometric information may be available for review by a clinician in real time as the PEMF treatment is being delivered to the patient. In some cases, PEMF level information and/or patient biometric data may be used as data to verify or validate clinical trials or verify that a patient has received PEMF therapy.

In some variations, a detected PEMF level may be transmitted to a separate device such as the PEMF therapy device 110, the mobile device 160 or the monitoring server 140. The detected PEMF level may then be compared to a predetermined PEMF level at the receiving device.

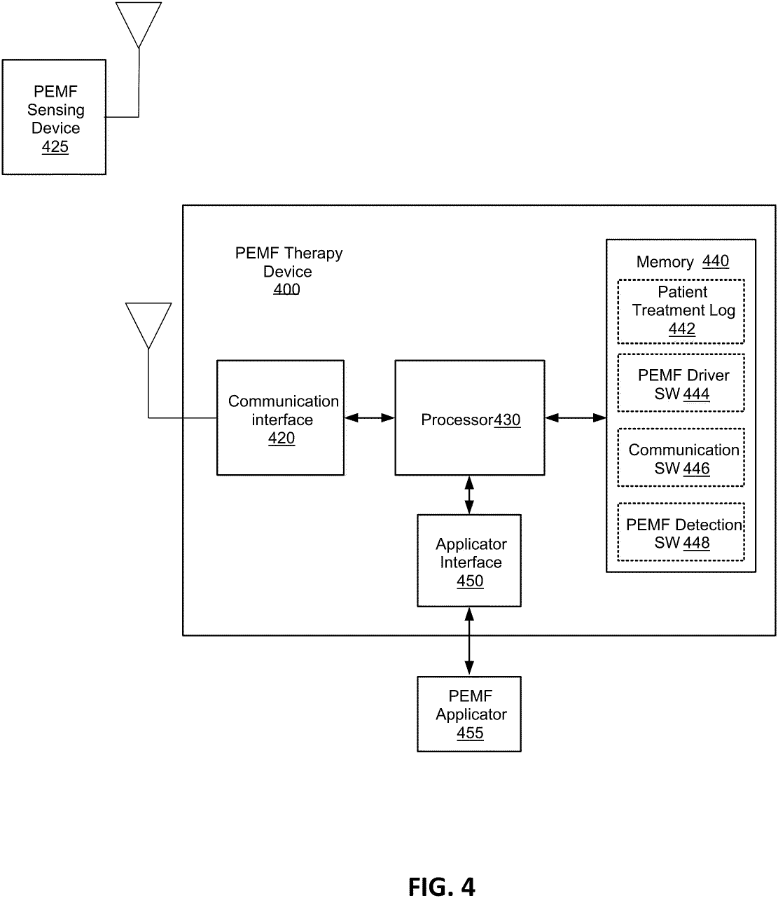

FIG. 4 shows a block diagram of a PEMF therapy device 400 according to some examples. The PEMF therapy device 400 may include a communication interface 420, a processor 430, a memory 440, and an applicator interface 450.

The communication interface 420, which is coupled to the processor 430, may be coupled to an antenna and include devices, components, and/or modules to provide wireless communication capabilities for the PEMF therapy device 400. For example, the communication interface 420 may transmit and receive wireless data according to Bluetooth, Wi-Fi, and/or cellular communication protocols.

The applicator interface 450, which is also coupled to the processor 430, may be used to interface and control any feasible PEMF applicator, such as PEMF applicator 455. The applicator interface 450 may provide a high-power pulsed electromagnetic field signal to the PEMF applicator 455. The PEMF applicator 455, in return, may emit an electromagnetic field, such as a magnetic field, that may treat and penetrate body tissues. In some examples, the applicator interface 450 may include driver circuitry (not shown) to generate the high-power pulsed electromagnetic field signals for any feasible PEMF applicator.

A PEMF sensing device 425, which may be an example of the PEMF sensing device 130 of FIG. 1 or the PEMF sensing device 200 of FIG. 2 may transmit data associated with detected PEMF signals to the PEMF therapy device 400 through the communication interface 420. In some examples, the PEMF sensing device 425 may also transmit patient biometric information to the PEMF therapy device 400 through the communication interface 420.

The processor 430, which is also coupled to the memory 440, may be any one or more suitable processors capable of executing scripts or instructions of one or more software programs stored in the PEMF therapy device 400 (such as within memory 440).

The memory 440 may include a patient treatment log 442 that may be used to locally store PEMF treatment information. For example, the patient treatment log 442 may include detected PEMF treatment times and duration, number of treatments per day information, detected PEMF treatment levels, patient biometric information, or any other feasible treatment information.

The memory 440 may also include a non-transitory computer-readable storage medium (e.g., one or more nonvolatile memory elements, such as EPROM, EEPROM, Flash memory, a hard drive, etc.) that may store the following software modules: a PEMF driver software (SW) module 444 to control the high-power pulsed electromagnetic field signal provided by the applicator interface 450; a communication SW module 446 to control transmitting and receiving data though the communication interface 420; and a PEMF detection SW module 448 to determine whether any PEMF data from the PEMF sensing device 425 is indicative of a PEMF therapy being delivered to a patient.

Each software module may include program instructions that, when executed by the processor 430, may cause the PEMF therapy device 400 to perform the corresponding function(s). Thus, the non-transitory computer-readable storage medium of memory 440 may include instructions for performing all or a portion of the operations described herein.

The processor 430 may execute the PEMF driver SW module 444 to control the energy signals delivered via the applicator interface 450 to one or more PEMF applicators (not shown). For example, execution of the PEMF driver SW module 444 may cause the applicator interface 450 to provide a high-power pulsed electromagnetic field signal. Execution of the PEMF driver SW module 444 may cause the applicator interface 450 to increase or decrease the PEMF therapy delivered through the applicator interface 450.

The processor 430 may execute the communication SW module 446 to communicate with any other feasible devices. For example, execution of the communication SW module 446 may enable the PEMF therapy device 400 to communicate via cellular networks, Wi-Fi networks conforming to any of the IEEE 802.11 standards, Bluetooth protocols put forth by the Bluetooth Special Interest Group (SIG), or the like. In some embodiments, execution of the communication SW module 446 may enable the PEMF therapy device 400 to communicate directly or indirectly with the PEMF sensing device 425.

The processor 430 may execute the PEMF detection SW module 448 to determine whether PEMF energy detected by the PEMF sensing device 425 indicates a PEMF therapy being received by the patient. For example, execution of the PEMF detection SW module 448 may compare a PEMF level detected by the PEMF sensing device 425 to a predetermined threshold. If the PEMF level is greater than the predetermined threshold, then the patient is receiving PEMF therapy. On the other hand, if the PEMF level is not greater than the predetermined threshold, then the patient may not be receiving PEMF therapy.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

### EXAMPLES

For example, described herein are apparatuses and systems configured to track patient compliance and/or non-compliance when using a pulsed electromagnetic field (PEMF) treatment apparatus, and/or for adjusting treatment based on the actual dose (treatment) received.

A prototype apparatus for detecting applied PEMF treatment was constructed. The apparatus (e.g., device, system, etc.) is configured to be worn by a patient around the patient’s wrist, much like a watch. The apparatus houses detection circuitry as well as a microcontroller. It also interfaces with a mobile application that the patient can download. This mobile application may be supported by iOS, Android, etc.

Although other configurations (not limited to a watch) may be used, in this example the watch design may be comfortably worn by the patient.

Once the device has been attached properly, the patient may turn the device on using a control (e.g., button, switch, etc.). The patient will then administer or otherwise receive a PEMF treatment. The detection circuitry may communicate with the microcontroller to detect, analyze and/or track the treatment. The microcontroller may communicate with the mobile app and alert the user if treatment has or has not been detected. In some examples the mobile app may analyze and/or track detected PEMF signals. The treatment information may be stored and/or transmitted (e.g., wirelessly) to a remote server (e.g., cloud server).

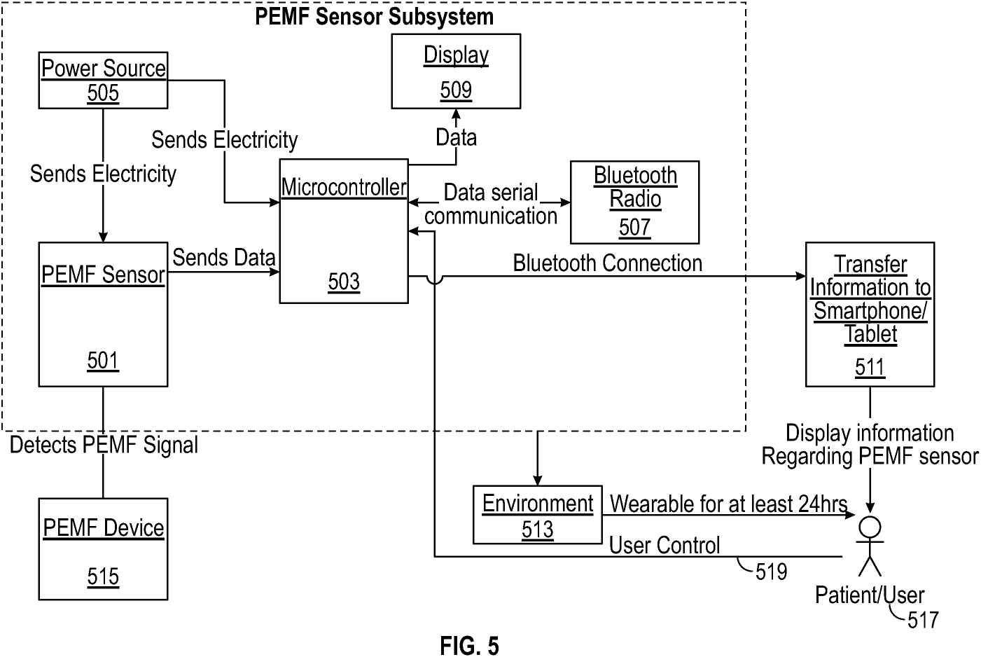

FIG. 5 is a schematic illustrating one example of an apparatus (e.g., PEMF sensor subsystem 519) as described herein. In this example the apparatus includes a PEMF sensor 501, in communication with a microcontroller 503, and a power source 505. The power source may include a battery, capacitor, etc. The microcontroller may communicate with (e.g., receive input/output) display 509, and may include one or more inputs for receiving input from the user 517 (including one or mor controls or control inputs) and/or a remote processor 511 (e.g., smartphone, tablet, etc.) via a wireless circuit, such as a Bluetooth radio circuit 507. The apparatus may generally be configured to detect the application of a PEMF signal from a PEMF device 515 and may coordinate detected signals with those applied by the PEFM device, in contrast to other external environmental signals 513.

The wearable PEMF sensor subsystem may generally detect the presence or occurrence of a PEMF therapy session within the treatment area, e.g., on the patient. As descried above, the PEMF therapy may be applied to the patient at a site that is remote from the sensing location (e.g., PEMF therapy applied to the lower trunk, such as feet, leg, etc.) may be detected using a wrist-worn or hand held apparatus. In general, the PEMF sensor subsystem may be wearable by the user for at least 24 hours. In some examples the PEMF sensor may connect to a host (e.g., smartphone, tablet, or PEMF therapy system) and transfer information bidirectionally, e.g., via Bluetooth. The system may display the raw data collected from the PEMF sensor, and/or processed data.

The PEFM sensor subsystems described herein may provide access to patient information and compliance and/or may provide feedback during or after the applied therapy to modify the applied PEMF therapy, based on the actual dose receive by the patient. The wearable PEMF sensor may be used within a clinical environment and/or outside of the clinical environment (e.g., at home).

In general, all user-accessible surfaces of the PEMF sensor subsystem are biocompatible.

The PEMF sensing sub-systems described in this example are configured to sample the presence of PEMF treatment at regular intervals (e.g., once per minute, twice per minute, four times per minute, six times per minute, 12 times per minute, every second, every 2 seconds, every 0.5 seconds, etc.). In some examples the PEMF sensor subsystem may be turned on by the user. In some examples the PEMF sensor subsystem is activated by the PEMF applicator, which may transmit an “on” signal to the PEMF sensor subsystem, and/or may alert the PEMF sensor subsystem to the start and/or finish of an applied PEMF therapy.

In any of these methods and apparatuses, The PEMF sensor subsystem may timestamp the samples automatically when the device is turned on and/or when a signal above a background threshold is detected. In some examples, the PEMF sensor subsystem continually sends data to the host as it samples. Alternatively, the PEMF sensor subsystem may store data and transmit in bursts or after receiving a request from a remote processor (e.g., the application software and/or a remote server).

The PEMF sensor subsystem may include a wireless communications circuit, such as a Bluetooth circuit. Bluetooth functionality of the PEMF sensor subsystem may be configured to alternate between sleep and active mode. In some examples, the Bluetooth functionality works off of 3.3 V - 5 V supply. In some examples, the PEMF sensor subsystem operates off of a small rechargeable battery. In some examples, a wearable PEMF sensor subsystem may be configured to be worn comfortably on the wrist. Other wearable devices may be worn on the neck (e.g., necklace), arm (e.g., armband), waist (e.g., belt, or belt attachment), finger (e.g., ring), head (e.g., hat, headband, etc., torso (e.g., attached to or part of a clothing, brooch, etc.), etc.

In one example the PEMF sensor subsystem is worn as a wrist-worn device, e.g., a watch. In general, the PEMF sensor subsystem may weigh no more than 100 grams. The sensor/device housing may have an area of less than or equal to 3.5 x 2 inches. The PEMF sensor subsystem may be functional in non-clinical environments (e.g., home wear, etc.).

The PEMF sensor subsystem may communicate with a mobile device (e.g., smartphone, tablet, etc.) and may include one or more outputs (e.g., LEDs, display, etc.). For example, the PEMF sensor subsystem may include a display indicating that it is on and/or that PEMF therapy is being applied, and/or the level or intensity or dose of PEMF therapy applied. Surprisingly, as PEMF therapy is often otherwise undetectable by the patient, output provided by the wearable PEMF sensor subsystem may be both helpful and therapeutically significant to the patient. Any appropriate output may be used, including an LED (lighting or changing lighting), video display, etc. as the PEMF energy is applied and detected. In some examples the output may be done by a user-held device (smartwatch, phone, tablet, laptop, etc.) instead or in addition to the PEMF sensor subsystem. For example, in some variations the PEMF sensor subsystem may communicate with an application software (e.g., a mobile app) and the software may display the presence and/or timestamp of the PEMF signal detected. Thus, in any of these examples, the PEMF sensor subsystem may provide a display of the PEMF signal detected. For example, the mobile app may display the presence of PEMF treatment based on the sample data.

In any of the PEMF sensor subsystem apparatuses described herein the apparatus may detect, store and/or transmit one or more environmental parameters in addition to the detected PEMF signal/therapy. For example, any of these apparatuses may include or detect environmental and/or patient temperature, humidity, time of day, etc.

The mobile app associated with the PEMF sensor subsystem may notify the patient to remove and/or turn off the PEMF sensor subsystem once the PEMF treatment is finished, and all data has been collected. Alternatively, the PEMF sensor subsystem may be configured to automatically shut down (and/or turn on) based on a signal from the PEMF applicator.

As mentioned above, the PEMF sensor subsystems described herein may include an output, such as one or more LEDs, a display, etc., and may be configured to indicate the detection of a PEMF signal. The apparatuses described herein may include one or more configuration items (e.g., software configuration items). In some examples the PEMF sensor subsystem may include a PCB, configured to detect the presence of PEMF, as well as a microcontroller to collect this data from the circuit board. Bluetooth may be involved in the transmission of data to an external source for a mobile application. Each of these components may work together to create the software subsystem as a whole. The mobile interface may include of a patient interface, administrator interface, and database for data collected by the microcontroller.

For example, any of the PEMF sensor subsystems described herein may include a printed circuit board (PCB) that will have the capability of detecting the presence of PEMF in a specific area of treatment. The circuit board will use an analog to digital converter to determine whether or not there was PEMF detected in the area. This data will then be sent to the microcontroller through a signal in an ADC port. Software will then be written for the microcontroller that will take this input from the PCB and send it to a mobile app via Bluetooth that will be displayed to the user as well as transmitted to a remote server for storage and/or analysis. Transmitted data may be associated with the patient (e.g., for use by the patient’s physician, nurse, or other medical professional, and/or for use by an insurer).

In some examples the PEMF sensor subsystem may include software that also includes a user/patient interface that includes a login page, account information page, and treatment page. An administrator interface may include a login page, account information page, and a patient data page.

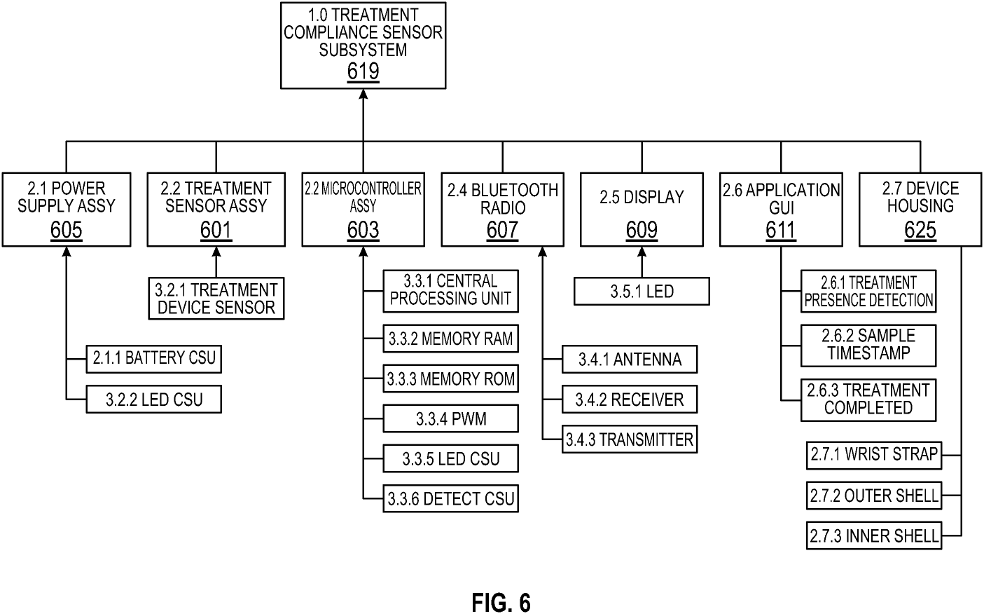

FIG. 6 schematically illustrates one example of the various component portions of one example of a PEMF sensor subsystem 619 as described herein. These subsystem components may each include control software. In this example, the PEMF sensor subsystem includes a power supply subassembly 605 (e.g., including a battery and LED) and associated control software. The PEMF sensor subsystem also includes a treatment sensor assay 601 (e.g., treatment device sensor) and microcontroller subassembly (e.g., one or more processors/CPUs, memory, e.g., RAM/ROM, power management circuit(s), LED control circuit(s), detection control circuit(s), etc.). The PEMF sensor subsystem may also include a communications circuit (e.g., Bluetooth radio 607, including an antenna, receiver/transmitter, etc.), and a display 609 (e.g., LED, screen, etc.).

In some examples the PEMF sensor subsystem includes an application graphical user interface (GUI 611), e.g., as part of an application software, that may be executed on a patient’s smartphone, tablet, etc. The application GUI 611 may include an output UI for treatment presence detection, sample date/time stamp, treatment completion and/or treatment progress (e.g., percent completion tracking/progress indicator, etc.).

In any of the PEMF sensor subsystems described herein the apparatus may include an outer housing (e.g., device housing 625) and may include a wrist strap, outer shell, inner shell, etc.

Any of the apparatuses described herein may accept input from the sensor (e.g., PCB board) detecting the PEMF treatment, and may then use an analog to digital converter to determine whether or not the treatment has been administered. This data may then be sent to a mobile app, e.g., via Bluetooth, to be displayed for the user. The data will also or alternatively be sent to the clinical supervisors in order to verify patient compliance. One input to the PEMF sensor subsystem may be the PEMF treatment. For example, the PCB may receive a signal via an antenna. The energy may be received as current/voltage. The microcontroller may accept the signal in an ADC port. The signal may be compared to different ranges. In some examples the microcontroller software may consist of a series of if statements to determine if the treatment was detected. The PEMF sensor subsystem and/or the app may display the level of treatment (detected/not detected) based on the level of PEMF detected from the ADC converter. The response time of the ADC may depend on the sampling rate, which may be preset and/or adjustable. For example, the sampling rate may be 4 times per minute. The timer for the microcontroller may handle the sampling rate. Any error handling may be displayed on the PEMF sensor subsystem and/or the app.

The PEMF sensor subsystem and/or app may include a database that may contain the data including any timestamps of when each sample was collected. For example four samples may be collected per minute of treatment. Along with this sampling timestamp data, it may also be paired with the data containing the detection of PEMF. At each sample, the PEMF sensor subsystem may determine whether an applied PEMF therapy was detected. Overall, each data point may contain a timestamp along with whether this presence of PEMF was detected or not and/or the level of PEMF detected (and/or a frequency of PEMF detected). The data will then be sent to an application so that it may be processed and/or viewed after collection and/or during treatment. This data may help to determine if the patient complied with the clinical prescription and/or if the proper treatment (e.g., dose) was received. In general, the user interface and/or software may include cybersecurity protocols in order to protect patient information.

The application software may support patient interaction and may operate on a patient device (e.g., smartphone) and the microcontroller of the PEMF sensor subsystem may communicate with the device via Bluetooth. In some examples, the data received from the microcontroller may be stored in the appropriate data structure (e.g., as an object) and displayed for the user. The data may go through a series of if statements in order to determine if the PEMF treatment data is sufficient to state the treatment is present. If not present, the PEMF sensor subsystem and/or app may display a message stating no treatment was detected. The mobile app constraints may be open ended; it may display a timestamp of the data and the data itself and may receive data from the microcontroller and be able to read it correctly.

The software may be able to quickly detect the presence of a power source (e.g., power on of the PEMF sensor subsystem). The Bluetooth functionality of the PEMF sensor subsystem may alternate between sleep and active mode. The Bluetooth device may be able to be turned off and on. The PEMF device may communicate with a mobile device via Bluetooth or any other appropriate wireless technique. In some examples, Bluetooth protocols may involve a controller stack to deal with critical radio interference and to deal with the data. The mobile app may display the timestamp of the sample. The software may store the timestamp in an appropriate data type (e.g., date object, custom object, string, etc.). The software may display the timestamp of the sample in a human readable way. The timestamp may be displayed as a string on the UI. The software of the mobile app may be able to accept data from the microcontroller. In some examples, the software may notify the patient to track the appropriate duration timer class with methods for remove the sensor once the PEMF treatment is finished and all data has been collected.of the treatment. Alternatively the PEMF sensor subsystem may automatically power down (which may save battery life) after no signal is detected for a predetermine or variable time (e.g., 3 minutes, 5 minutes, 7 minutes, 10 minutes, etc.) and/or if a signal is received from the PEMF treatment device. In any of these PEMF sensor subsystems the apparatus may indicate, e.g., using LEDs, the detection of PEMF and/or contact with the skin, and/or that treatment is not detected.

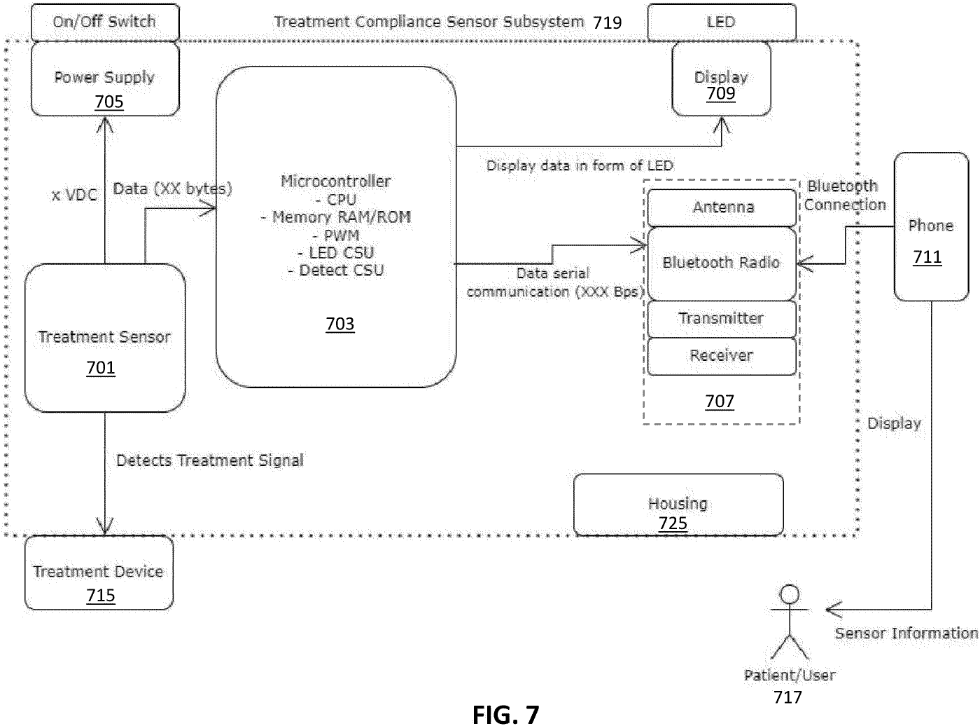

FIG. 7 illustrates another example of a schematic of a PEMF sensor subsystems apparatus 719, similar to that shown in FIG. 5. In this example the PEMF sensor subsystems (“treatment compliance sensor subsystem”) includes the treatment sensor 701, power supply 705 (including a control input, such as an on/off switch), and a controller 703 (microcontroller, e.g., CPU, memory, PWM, LED CSU, Detect CSU, etc.). The PEMF sensor subsystem also includes one or more outputs (e.g., display 709) and wireless circuitry 707. In FIG. 7 the wireless circuitry may be Bluetooth circuitry, and includes one or more antenna, radio circuitry and transmitter/receiver (or in some cases transceiver). All or some of these components may be enclosed within a housing 725, which may be configured to be worn, e.g., on a patient’s wrist.

The PEMF sensor subsystem may communicate with a remote device or server, including but not limited to a patient’s 717 smartphone 711, etc. In some examples the PEMF sensor subsystems may also or alternatively communicate with the PEMF treatment device 715.

### Smartwatch and Smartphone Based PEMF Sensor Subsystems

As mentioned above, any of the methods and apparatuses described herein may include software (e.g., an application or “app”) that may communicate with, control, and/or analyze the information from a PEMF sensor subsystem. In some examples the PEMF sensor subsystem may be configured as software running on an existing smartwatch and/or smartphone that uses the onboard sensing/receiving circuity to detect a signal from the patient indicating the application of a PEMF treatment.

For example, personal electronics such as (but not limited to) smartphones and smart watches may include software that is configured to detect an applied PEMF treatment when worn and/or held against or held by the patient. In general, a worn or hand-held electronics such as a smartphone or smart watch may include a charging circuitry that operates at 13.56 MHz. 13.56 MHz is a sub harmonic of the frequency that may be used for PEMF. For example a PEMF applicator apparatus may use a carrier frequency of about 27.12 MHz. The charging circuity of the hand-held electronics apparatus may rectify a received RF signal to a DC signal that can charge the device. These hand-held devices may include sensing circuitry that senses the received RF signal and/or the rectified DC signal in order to alert the device that a signal (usually a charging signal) is detected and to enable the charging circuitry.

In some examples, software running on the hand-held device may instead configure the device to detect this signal and analyze it to determine if a PEMF signal (e.g., applied during a treatment) is present or not. For example, application software (an “app”) may be installed on the hand-held or worn device, such as a smartphone or smart watch, and may configure the devic to monitor the detection signal from the charging detection circuit. In some cases the PEMF radio-frequency signal is smaller than what a typical wireless charger generates, however, the sensing circuity may be sufficient. Alternatively of additionally, the device may be modified to include an amplification circuit to improve sensitivity to the signal. In some examples a custom housing may be provided for the device (e.g., smartphone/smart watch) while utilizing the internal circuitry of the device. For example, in some cases a housing, case, or other attachment may be used and configured to attach to the device and may include an amplifier (e.g., amplification circuit).

Alternatively or additionally, a wearable and/or handled device may include an electrode or sensor (e.g., an ECG sensor) that may be used to detect the PEMF signal. For example, a wearable device configured to sense or detect an ECG signal may be configured, e.g., by a software (app) to detect a 27.12 MHz signal indicating PEMF application as described herein. In any of these example the device may be configured (e.g., by the software, etc.) to detect a signal within a 200 Hz to 1 KHz range in order to detect a pulse within this range which may also be associated with an applied PEMF treatment therapy. The software may indicate if a treatment is applied and/or the duration of treatment applied.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/- 0.1% of the stated value (or range of values), +/- 1% of the stated value (or range of values), +/- 2% of the stated value (or range of values), +/- 5% of the stated value (or range of values), +/- 10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for detecting a pulsed electromagnetic field (PEMF) treatment provided to a treatment site on a patient, the method comprising:
detecting, with a PEMF sensor that is held or worn at a detection site on the patient that is distal from the treatment site, an electrical signal;
determining that the electrical signal correspond to a PEMF treatment applied to the treatment site; and
transmitting, to a second device, data characterizing the PEMF treatment in response to determining that the electrical signal corresponds to a PEMF treatment level.

2. The method of claim 1, wherein determining the electrical signal corresponds to the PEMF treatment applied to the treatment site comprises determining that the electrical signal is greater than a threshold.

3. The method of claim 1, wherein determining the electrical signal corresponds to the PEMF treatment applied to the treatment site comprises determining that the electrical signal has a frequency within a predefined range.

4. The method of claim 1, wherein transmitting data characterizing the PEMF treatment comprises transmitting one or both of: a PEMF treatment level and a PEMF treatment duration.

5. The method of claim 1, wherein the PEMF treatment level is proportional to pulsed electromagnetic fields provided by a PEMF applicator.

6. The method of claim 1, wherein the PEMF sensor is held or worn at the detection site on the patient’s arm, hand or wrist, or on a garment worn by the patient.

7. The method of claim 1, wherein the PEMF sensor is one of: an armband, a ring, a belt, a wrist band, a necklace, a strap, a band, an anklet, a disposable patch, a smart watch adaptor, a watch adaptor, or a combination thereof.

8. The method of claim 1, further comprising concurrently providing PEMF treatment by the second device.

9. The method of claim 1, wherein the second device is a PEMF therapy device coupled to a PEMF applicator.

10. The method of claim 1, wherein the second device is at least one of a smart phone, a tablet computer, a laptop computer, a smart watch, or a server computer.

11. A pulsed electromagnetic field (PEMF) detection apparatus, the apparatus comprising:
a PEMF sensor comprising one or more coils;
a housing configured to support the PEMF sensor, further configured to be held or worn by a patient; and
a processor configured to determine that a patient is receiving a PEMF treatment, the processor, a memory coupled to the processor, the memory configured to store computer-program instructions, that, when executed by the processor, perform a computer-implemented method comprising:
detecting an electrical signal with the PEMF sensor;
determining that the electrical signal corresponds to a PEMF treatment applied to a treatment site on the patient’s body; and
transmitting, to a second device, data characterizing the PEMF treatment in response to determining that the electrical signal corresponds to the PEMF treatment.

12. The apparatus of claim 11, wherein the processor is configured to determine that the electrical signal correspond to the PEMF treatment applied to the treatment site on the patient’s body by determining that the electrical signal is greater than a threshold.

13. The apparatus of claim 11, wherein the processor is configured to determine that the electrical signal correspond to the PEMF treatment applied to the treatment site on the patient’s body by determining that the electrical signal has a frequency within a predefined range.

14. The apparatus of claim 11, wherein transmitting data characterizing the PEMF treatment comprises transmitting one or both of: a PEMF treatment level and a PEMF treatment duration.

15. The apparatus of claim 11, wherein transmitting data characterizing the PEMF treatment comprises transmitting a magnitude and/or a frequency of the detected electrical signal.

16. The apparatus of claim 11, wherein the housing is configured to be held or worn by the patient’s arm, hand or wrist.

17. The apparatus of claim 11, wherein the housing is configured to be worn on a garment worn by the patient.

18. The apparatus of claim 11, wherein the housing is configured to be an armband, a ring, a belt, a wrist band, a necklace, a strap, a band, an anklet, a disposable patch, a smart watch adaptor, a watch adaptor, or a combination thereof.

19. The apparatus of claim 11, further comprising a wireless transmitter.

20. A pulsed electromagnetic field (PEMF) detection apparatus comprising:
a PEMF sensor configured to be worn or held at a detection site to detect an electrical signal indicative of a PEMF treatment received by a patient at a site remote from the detection site;
a comparison unit configured to determine that a detected electrical signal is greater than a PEMF threshold and to determine data characteristic of the PEMF treatment; and
a wireless transmitter configured to transmit the data characteristic of the PEMF treatment to a second device in response to a determination that a detected PEMF level is greater than a threshold.
